(19) United States
(12) Patent Application Publication
(10) Pub. No.: US 2025/0177636 A1
(43) Pub. Date: Jun. 5, 2025

(54) MECHANICAL CONNECTOR FOR INJECTION DEVICES

(71) Applicant: Sanofi, PARIS (FR)

(72) Inventors: Brunhilde GOOTZ, Frankfurt am Main (DE); Thomas KLEMM, Frankfurt am Main (DE); Heiko RACKEL, Frankfurt am Main (DE)

(21) Appl. No.: 18/843,946

(22) PCT Filed: Mar. 9, 2023
(86) PCT No.: PCT/EP2023/055987

§ 371 Date: Sep. 4, 2024

(30) Foreign Application Priority Data
Mar. 11, 2022 (EP) 22315057.4

(51) Int. Cl. A61M 5/00

(52) U.S. Cl. CPC A61M 5/008

(57) ABSTRACT

Mechanical Connector for Injection Devices The present disclosure relates to a connector (100) for establishing a mechanical connection between a first injection device (1) and a second injection device (11), wherein the first injection device (1) and the second injection device (11) comprises a housing (20) of elongated shape extending in a longitudinal direction (z), the connector (100) comprising: —a body (101; 201; 301; 401; 501; 601), —a first connecting portion (110; 210; 310; 410; 510; 610) connectable to the first injection device (1) or integrated into the first injection device (1), —a second connecting portion (120; 220; 320; 420; 520; 620) detachably connectable to the second injection device (2).

### FIELD

The present disclosure relates to the field of injection devices, in particular to handheld injection devices, such as injection pens. In a further aspect the present disclosure relates to a connector for establishing a mechanical connection between a first injection device and a second injection device. In a further aspect the disclosure relates to an injection device provided with a connector for establishing a mechanical connection to another injection device. In another aspect the present disclosure relates to a kit comprising a first and a second injection device and further comprising a connector for mutually interconnecting the first and the second injection devices. In still another aspect the disclosure relates to a method of interconnecting a first injection device and a second injection device by making use of a connector.

### Background

Drug delivery devices allowing for multiple dosing of a required dosage of a liquid medicinal product and further providing administration of such liquid drug to a patient, are as such well known in the prior art. Generally, such devices have substantially the same purpose as that of an ordinary syringe. Typically, a medicinal product to be administered is provided in a cartridge having a moveable piston or bung mechanically interacting with a piston rod of a drive mechanism of the drug delivery device. By applying thrust to the piston, a certain and pre-defined amount of the medicinal fluid is expelled from the cartridge.

Some injection devices are implemented or provided as a pre-filled injection device. They may have to be stored in a refrigerated area. Injection devices, such as handheld pen injectors or the like injection devices may be implemented or manufactured as a disposable device, that is intended to be discarded after use. Insofar, a patient regularly using such injection devices and storing numerous injection devices has to provide sufficient refrigerated storage space. Moreover, also transportation and storage of a large number of individual injection devices in the product chain from manufacturing to commercial distribution of such injection devices requires an intelligent and space saving packaging of such injection devices.

Providing and operating rather large refrigerated storage spaces is rather energy extensive. It is therefore desirable to optimize storage space for accommodating or storing injection devices, such as a pen-type injectors. It is a particular aim of the present disclosure to reduce storage space required for storing a given number of injection devices. Moreover, it is intended to reduce material and costs for a packaging of injection devices. It is thus intended to provide a new approach on how to store and/or commercially distribute a large number of injection devices in a rather practical, intuitive and storage-space saving way.

### SUMMARY

In one aspect the present disclosure relates to a connector for establishing a mechanical connection between a first injection device and a second injection device. Here, the first injection device and the second injection device each comprise a housing of elongated shape extending in a longitudinal direction. The longitudinal direction, which may be also denoted as an axial direction may define a longitudinal distal direction and an oppositely facing longitudinal proximal direction. The injection device may comprise a dispensing end facing in distal direction. Some injection devices may comprise a dose button and/or trigger button at or near their oppositely located proximal end.

With some examples at least one of the first injection device and the second injection device is implemented as a pen-type injector. With further examples, the first injection device and the second injection device are both implemented as a pen-type injector, e.g. of equal or substantially equal dimensions or size.

The connector comprises a body. The connector and/or the body comprise a first connecting portion, which is connectable to the first injection device or which is integrated into the first injection device. The connector further comprises a second connecting portion detachably connectable to the second injection device. With some examples the first connecting portion is operable and configured to mechanically connect the body of the connector to the first injection device, typically to a housing component of the first injection device. By way of the second connecting portion, which may be fastened or integrated into the body, there can be provided a detachable connection, hence a detachable mechanical connection to the second injection device. In this way and by making use of the connector the first and the second injection devices can be mutually interconnected.

In the same way and by using a further connector the second injection device can be further connected to a third injection device. By making use of a third connector the third injection device can be connected to a fourth injection device and so on. In this way, the connector and/or a number of connectors can provide a mutual mechanical and releasable interconnection of a plurality of individual injection devices, thus facilitating the overall handling and packaging of a respective number of injection devices.

In this way, packaging material for individually wrapping or packaging the first injection device and/or the second injection device can become superfluous. Moreover, by way of the connector, the first injection device and the second injection device can be arranged in a comparatively large storage space, wherein the individual first, second and some optional further injection devices are mutually kept in a rather stable position or orientation relative to each other. Here, the individual device do no longer have to be individually arranged in a separate packaging.

The mutually interconnected first and second and optional further injection devices may be therefore inherently protected against damages caused by an uncontrolled tumbling of injection devices that could otherwise arise when a large number of injection devices mutually collide when loosely arranged in a comparatively large storage space.

By mutually fastening the first and the second injection devices relative to each other there can be provided a kit or an array of injection devices, which is dimensionally stable and which can be easily arranged in a comparatively large packaging or storage space. Any mechanical shock applied to the storage space, e.g. to an outer packaging, e.g. during storage and/or transportation can be transferred to the entire arrangement of injection devices but will not invoke a relative movement among the injection devices. Due to the mutual fastening of the individual injection devices mutual collisions or uncontrolled tumbling of injection devices inside a comparatively large storage space or packaging can be effectively prevented.

Moreover, by directly interconnecting the first and the second injection device by making use of a connector the total volume occupied by the first and second injection devices can be reduced compared to a configuration, in which the first injection device is arranged in a first packaging and in which the second injection device is arranged in a second packaging. Insofar and by making use of one or several connectors for interconnecting first, second and optional third or fourth injection devices, the total storage space required for storing the respective number of injection devices can be reduced and minimized compared to solutions, in which the first injection device is arranged in a first packaging and wherein a second injection device is arranged in a second packaging.

Moreover, and by mutually interconnecting first and second injection devices in a releasable or detachable way not only storage space but also packaging material required for packaging of the injection devices can be reduced. Insofar, the connector enables an ecologically friendly and resource saving arrangement for storing and transporting a large number of injection devices. Reducing storage space for a number of injection devices is of particular benefit when the injection devices have to be stored in a refrigerated area. Hence, existing storage space can be accommodated with an increased number of injection devices. In another aspect, less storage space is required for storing the same amount of injection devices.

According to a further example the first connecting portion and the second connecting portion of the connector are rigidly fastened to one another. With some examples the first connecting portion is a part of the body of the connector. With further examples also the second connecting portion is a part of the body of the connector. With some examples the first connecting portion and the second connecting portion are immovably attached to one another. They may be immovably connected or rigidly fastened to one another. In this way and when the first connecting portion is connected to the first injection device and when the second connecting portion is connected to the second injection device the rather rigid or mechanically stiff implementation of the body allows and provides a rather immobile arrangement of the first injection device relative to the second injection device. The first and the second injection devices can be kept in a rather dimensionally stable configuration by way of the connector.

According to a further example the body is a unitary body and/or the body is unitarily formed. The first connecting portion and the second connecting portion may be integrally formed with the body. A unitary or single pieced implementation of the body and/or of the entire connector allows for a rather simple and cost-efficient implementation thereof. With some examples the body and/or the entire connector may be made of an injection molded material, e.g. an injection molded plastic material.

According to another example at least one of the first connecting portion and the second connecting portion comprises a fastening bracket configured to mechanically engage with a housing or housing portion of at least one of the first injection device and the second injection device. The fastening bracket may provide a positive interlock with the housing or housing component of at least one of the first injection device and the second injection device. The fastening bracket may comprise a clip fastener or a clip joint by way of which the first connecting portion or the second connecting portion can be clip-fastened to the housing or to the housing portion of the first or second injection device. The fastening bracket may be configured to engage with the housing or housing portion of the injection device by forming a positive connection or positive interlock.

With some examples not only one but both connecting portions comprise a fastening bracket. Here, a first fastening bracket may be attachable or connectable to the housing or housing portion of the first injection device and the second fastening bracket may be detachably connectable to the housing or to the housing portion of the second injection device. With further examples, wherein the first connecting portion provides a connection of the connector to the first injection device and wherein the second connecting portion provides a detachable connection to the second injection device the connector can be provided as a separate part. In this way existing injection devices can be retrofitted with a connector to provide a mutual interconnection between first and second injection devices.

With some examples the first connecting portion may be somewhat identically shaped to the second connecting portion. In this way, the first connecting portion can be connected to a first portion of the housing or housing portion of the first injection device and the second connecting portion can be attached or connected to the same portion of the housing or housing portion of the second injection device.

When the first and the second connecting portions are somewhat identically or symmetrically shaped there may be at least one or two attachment configurations for attaching the connector to the first and the second injection devices. According to one example the first connecting portion may be connectable to the first injection device while the second connecting portion may be connectable to the second injection device. With another configuration it is the second connecting portion that is attachable or connectable to the first injection device whereas the first connecting portion of the connector is attachable or connectable to the second injection device.

According to another example the first connecting portion and the second connecting portion may be differently shaped. With some examples the first connecting portion comprises a fastening bracket of a first dimension and the second connecting portion comprises a second fastening bracket of a second dimension that distinguishes from the first dimension. Here, the first connecting portion and hence the first fastening bracket is configured to attach to a first portion of the housing or housing portion of the first injection device, whereas the second connecting portion and hence the second fastening bracket is configured to attach or to connect to a second portion of the housing or housing portion of the second injection device.

Here, the first and second portions of the housing or housing portions may be separated in longitudinal direction. This may require or enforce a connection between first and second injection devices, which is only enabled and possible when the first injection device is in a predefined orientation relative to the second injection device. The elongated first and second injection devices may have to be arranged radially flush and parallel to each other. Here, radially flush means that the proximal and/or distal longitudinal ends of a number of injection devices interconnected to each other by one or numerous connectors are flush in radial direction, i.e. transverse to the longitudinal direction.

With other configurations it is conceivable, that the first injection device is inverted by 180° relative to the second injection device with regard to an axis of rotation extending in radial direction through a longitudinal center of the first injection device. With injection devices comprising a substantially tubular shaped housing part, wherein e.g. a distal end of the injection device has an outer radial dimension that differs from the outer radial dimension of the proximal end, such a pairwise inverted orientation of first and second injection devices may further help to reduce the storage space required for arranging first and second and even further injection devices in a densely packed configuration.

With some examples the fastening bracket is dimensionally stable. It may be rather stiff and/or rigid. With other examples the fastening bracket may be resiliently deformable. It may exhibit a certain degree elasticity by way of which a fastening configuration, such as a clip-type fastening or fixing to a housing or housing component of any of the injection devices can be provided.

According to a further example the fastening bracket is of circular, semicircular or concave shape. With a circular or annular shape the fastening bracket may be configured for mounting onto the housing of the injection device along a longitudinal direction. Here, the fastening bracket, e.g. the annularly shaped fastening bracket may be configured to receive a distal or proximal end of the housing of the first or second injection device. The fastening bracket may comprise an inner diameter or cross-section that is slightly larger than at least one of the distal or proximal end of the housing of the injection device or device component. This way, the injection device or housing thereof can be inserted in longitudinal direction into the circularly or annularly shaped fastening bracket until the fastening bracket engages a radially widened portion, e.g. in the middle section or at a predefined distance from one of the proximal or distal end of the injection device. In the region of the radially widened portion the housing of the injection device comprises a larger diameter or cross-section compared to the distal or proximal end. The larger diameter closely matches the inside cross-section or diameter of the fastening bracket. In effect, that can be provided a friction fit or a positive sitting between an inside of the fastening bracket and an outside surface of the housing or housing section of the injection device.

With other examples and when the fastening bracket is of semicircular shape, it may be fastenable to the e.g. tubular shaped housing of the injection device in radial direction, e.g. by way of a clip fastening routine. Here, the semicircular shape of the fastening bracket may comprise a circular or circumferential extent of more than 180° and less than 270°, thereby allowing for a clip-type fastening procedure, during which the fastening bracket is at least temporally deformed to receive the housing or housing component of one of the first and the second injection devices along a radial direction. With other examples, the semicircular shape or extension may comprise a circumferential extent of more than 270°. Here, the fastening bracket may be configured for receiving the housing or housing component in longitudinal direction.

The fastening bracket, in particular its resiliently deformable sections may provide a kind of a clamping when the housing or housing component is inserted therein in radial or longitudinal direction.

With some examples the housing or housing portion of the injection device may comprise a circumferential groove matching in size and shape with the fastening bracket of the connector. In this way, that can be provided a kind of a positive fit and/or a friction fit between the fastening bracket of any of the first and second connecting portions and the housing of the injection device.

According to another example the fastening bracket comprises a receptacle sized to receive at least a part of the housing or housing portion of at least one of the first injection device and the second injection device. For instance, the fastening bracket may be configured to clasp around the outside surface of a sidewall portion of a tubular shaped housing component, such as a body or cartridge holder of the housing of the injection device. With other examples the fastening bracket may be sized to receive a protective cap of the housing of the injection device. With still another example the fastening bracket and/or the receptacle formed by the fastening bracket may be sized to receive a fastening clip of a protective cap of the injection device.

In effect, there may be provided numerous implementations of first and second connecting portions and respective fastening brackets, which may be individually shaped to receive a particular component or portion of a housing or housing portion of the injection devices.

According to a further example the fastening bracket comprises a first arm portion and a second arm portion configured to at least partially circumferentially enclose the housing or housing portion of at least one of the first injection device and the second injection device. The first arm portion and/or the second arm portion may be of arcuate shape. The first arm portion and the second arm portion may be interconnected by a middle section of the fastening bracket. The first arm portion may comprise a free end facing away the middle section. The second arm portion may also comprise a free end facing away the middle section.

The first arm portion and the second arm portion may be integrally formed with the middle section. Hence, the first arm portion may extend or protrude away from the middle section along a first circumferential direction and the second arm portion may face away or extend away from the middle section along a second circumferential direction opposite to the first circumferential direction. The free ends of the first arm portion and the second arm portion may face towards each other. They may be separated by a slit or gap, thus forming a kind of an open annular structure of the fastening bracket. Such a configuration is of particular benefit when the fastening bracket is intended for a clip-fastening, i.e. for fastening to the housing or housing portion of the injection device along the radial direction, i.e. perpendicular to the longitudinal extent of the movement injection devices.

According to a further example the first arm portion and the second arm portion comprise a c-shaped profile in the transverse plane extending perpendicular to the longitudinal direction. With other examples the first arm portion and the second arm portion comprise a L-shaped profile. In either way, the first arm portion and the second arm portion are shaped to form an enclosing structure by way of which the housing or housing portion of the injection device can be received in the space formed between the first and the second arm portions in order to provide a clamping effect or a positive interlock with the fastening bracket. Typically, the free ends of the arm portions facing away the middle section extends towards each other.

According to a further example the first arm portion and the second arm portion comprise a free end section, e.g. facing away the middle section. The free end section may be provided with a radially inwardly extending flange portion. Here, radially inwardly means an extension with regards to their circumferential extent of the first and/or second arm portions. The flange portions of the first arm portion and the second arm portion may face towards each other so as to form e.g. an undercut to engage with a housing or housing portion of the respective injection device.

With a further example the fastening bracket comprises a closed ring structure. The closed ring structure provides a comparatively large stability and rigidity to the fastening bracket. With other examples the fastening bracket comprises an open ring structure. Here, the ring structure is interrupted or intersected by a through opening or through recess. Here, first and second arm portions are separated by the through recess extending longitudinally through the ring structure. Here, the fastening bracket shows and exhibits an increased degree of flexibility. It may enable a clip type fastening, i.e. a fastening in a radial fastening direction.

With another example the connector further comprises a base portion rigidly fastened to the first connecting portion and to the second connecting portion. The first connecting portion extends in a first radial direction from the base portion with regards to the longitudinal direction. The second connecting portion extends or protrudes in a second radial direction from the base portion. With some examples the first radial direction extends opposite to the second radial direction. With other examples the first radial direction extends at a predefined angle with regards to the second radial direction, e.g. perpendicularly.

When the first radial direction and the second radial directions face in opposite directions the first and the second injection devices, which are configured to mutually attach or connect by way of the first and second connecting portions, may be arranged in a longitudinal row, e.g. extending in radial direction. Here, a large number of injection devices could be arranged next to each other with their proximal and distal end portions flush as seen in radial direction, i.e. perpendicular to the longitudinal direction. The connectors may then act as radial connectors.

Here, the base portion may be located at a radial offset from the first injection device when the first connecting portion is engaged with the first injection device. The base portion may be also arranged radially offset from the second injection device when the second connecting portion is interconnected with the second injection device. In effect, with first and second radial directions extending in opposite directions, the base portion of the connector is located between adjacently disposed first and second injection devices.

The base portion may provide a kind of a distance spacer between the first and the second injection devices, thus keeping the first and second injection devices in a well-defined or predefined radial distance to each other. With some examples, the base portion may be rather tiny. It may comprise a radial extent of less than 1 cm, less than 5 mm, less than 3 mm or even less than 2 mm, thereby providing a rather densely packed configuration or mutual arrangement of first and second injection devices when mutually interconnected by a connector.

According to a further example the base portion is located between the first connecting portion and the second connecting portion. At least one of the first connecting portion and the second connecting portion extends or protrudes radially outwardly from the base portion. The first and the second connecting portions may be therefore arranged at a certain radial offset. This way, and when the first connecting portion is interconnected with the first injection device and when the second connecting portion is interconnected or fastened to the second injection device the first and second injection devices can be kept at a well-defined distance from each other in a collision-less manner.

According to another example, one of the first connecting portion and the second connecting portion comprises a male connecting part configured to detachably engage with a complementary shaped female connecting part of another one of the first connecting portion and the second connecting portion of another connector. The connector and the another connector may be identical connectors. With some examples also a housing portion of one of the first injection device and the second injection device may provide one of a male connecting part and a female connecting part configured to connect to a male or female connecting part of the connector.

With some examples the male connecting part may protrude radially outwardly from the body or from the base portion of the connector. With further examples it is the female connecting part thereof that protrudes radially outwardly from one of the body and the base portion of the connector. With further examples, the female connecting part may be configured as a recessed portion of the body or base portion that is sized to engage with the male connecting part of the connector.

With further examples, the male connector part and the female connector part both protrude radially outwardly from at least one of the body and the base portion of the connector. With some examples the male connecting part extends along the first radial direction and the female connecting part may extend radially outwardly along the second radial direction. Radially outwardly extending or protruding female and male connecting parts enables a rather easy and intuitive mutual fastening of the respective connectors.

Providing of a male connecting part and a female connecting part may be particularly intended with connectors, wherein the body or the base portion of the connector is integrated into a housing or housing portion of the injection device. Here, the entire connector may be integrated into such a housing or housing portion or housing component of an injection device. With such an integrated solution, it is no longer required to provide a separate connector in addition to the first and second injection devices.

According to a further example the base portion comprises a third connecting portion configured to detachably connect to a housing or housing portion of an injection device. The first connecting portion is configured to engage with the second connecting portion of another, e.g. identically shaped connector. Here, the third connecting portion may be exclusively configured for providing an attachment of the connector to the housing or housing portion of the first or second injection device.

Injection devices each equipped with such a connector may be then mutually interconnected by bringing the first connecting portion of the connector into engagement with the second connecting portion of another connector attached to another injection device. This solution is particularly applicable for retrofitting existing injection devices with a connector. With typical examples the third connecting portion comprises a fastening bracket, e.g. as described above in connection with the first and the second connecting portions.

Typically, the fastening bracket of the third connecting portion comprises an open or a closed ring structure, of e.g. annular shape so as to enclose or to clasp around the outer circumference of a housing or housing portion of the injection devices.

According to a further example the body of the connector is integrated into a housing or housing portion of the injection device. In other words, a housing portion or housing of the injection device may be provided with an integrated connector as described above. Insofar, any features, effects and benefits described above in connection with the connector may equally apply to a housing or housing component of an injection device provided with an integrated connector.

With some examples the connector may be integrated into a protective cap detachably connectable to a body or cartridge holder of an injection device. With other examples the connector may be integrated or attached to a body of a housing of an injection device.

With further examples and wherein the body is integrated into the protective cap of an injection device the first connecting portion may be integrated into a fastening clip of the protective cap. Here, the first connecting portion may be implemented as a male connecting part configured to engage with a complementary shaped female connecting part. With some examples an opposite or some other portion of the sidewall of the protective cap of the injection device may be provided with a female connector, e.g. implemented as the second connecting portion of such a connector. Here, the protective cap may form or constitute the connector as described herein.

According to a further example the second connecting portion comprises a connecting surface complementary to a counter connecting surface of a second connecting portion of another connector and detachably connectable to the counter connecting surface. Here, the connector comprises a dedicated connecting surface at its second connecting portion. The connecting surface faces outwardly when the connector is attached to the housing or housing portion, e.g. by a first connecting portion.

When the connector is integrated into a respective housing component the second connecting surface may likewise also face outwardly, e.g. radially outwardly from the respective housing component. The counter connecting surface of a second connecting portion of another connector may be particularly adapted to make contact and to interconnect to the connecting surface of the connecting portion of the first connector.

With some examples the connecting surface of the connector may comprise a hook and loop fastener. The counter connecting surface of another second connecting portion of another connector may comprise a complementary-shaped or complementary configured hook and loop fastener.

This way, and simply by bringing the connecting surface of a first connector in abutment or engagement with the complementary shaped counter connecting surface of second, and hence another connector there can be provided a mutual fastening and mechanical connection between the first and the second connectors, each of which attached or connectable to a respective housing or housing component of a first injection device and a second injection device.

With further examples, at least one of the first connecting portion and the second connecting portion of the connector may be sized and shaped to detachably connect to a housing of an add-on device or auxiliary device. Such an add-on device or auxiliary device may be detachably connectable to any of the first injection device and the second injection device. The add-on device may comprise at least one of a detector or sensor to detect setting and/or dispensing of a dose. The detector or sensor may be further configured to quantitatively measure the size of a dose currently set or dispensed. The add-on device or auxiliary device may comprise an electronic circuit, e.g. including a processor and an electronic storage, e.g. operatively engaged or connected to the sensor or detector. This way, injection-related data about the time and amount of a dose currently set or dispensed can be electronically stored.

With some examples, such an add-on device or auxiliary device further comprises a communication interface in order to exchange data with an external electronic device, such as a smartphone or tablet computer. With further examples the add-on device or auxiliary device comprises a display by way of which information can be visually illustrated. Typically, the add-on device or auxiliary device comprises at least one actuator, e.g. implemented as a button, thus allowing a user to control or to manipulate at least one of a configuration and an operation mode of the add-on device or auxiliary device.

Typically, such auxiliary devices or add-on devices comprise a housing suitable for mechanically interconnecting with at least one of the first connecting portion and the second connecting portion of the connector.

In this way, the connector can be used to mutually interconnect first and second injection devices, e.g. during transportation and/or storage. Once the injection devices are intended to be used by a patient, one of the first and the second injection devices can be detached from the connector and the add-on device or auxiliary device can be attached to the connector instead, thus providing a mutual connection of the other one of the first and second injection devices and the add-on device or auxiliary device. In this way, the connector can fulfill a twofold function. It can be universally used for mutually interconnecting a first and a second injection device and/or for interconnecting one injection device with an add-on device or auxiliary device.

Generally, and with regards to the functionality and/or usability of the connector one of the first injection device and the second injection device can be replaced by a housing of an add-on device or auxiliary device. Insofar, any references, technical effects and benefits described herein with regard to the mutual interconnection of first and second injection devices may equally apply to a mutual connection of an injection device with an add-on device or auxiliary device.

According to a further aspect the present disclosure relates to an injection device configured for setting and dispensing or injecting of a dose of a medicament. The injection device may be implemented as a handheld injection device, e.g. as a pen-type injector.

The injection device comprises a housing. The housing is configured or sized to receive a medicament container containing the medicament. The housing comprises at least one housing component. With some examples the housing comprises a tubular-shaped housing component comprising a tubular-shaped sidewall defining or confining an interior volume. The interior volume is typically occupied by the medicament container, e.g. implemented as a cartridge and by a drive mechanism operably engageable with the medicament container.

The injection device further comprises a drive mechanism operably engageable with the medicament container for expelling or withdrawing a dose of the medicament from the medicament container. Typically, the drive mechanism comprises a piston rod operable to exert a dispensing force onto a stopper or bung movably disposed inside the medicament container, typically inside a tubular-shaped barrel of the medicament container.

With other examples the drive mechanism may be implemented and configured to withdraw the dose of the medicament by way of suction from the interior of the medicament container.

The injection device further comprises a connector as described above. The connector is either connectable to one of the housing or to a housing component of the injection device. Alternatively, it is integrated into the housing or housing component of the injection device. Typically, the connector, which comprises a first connecting portion is connectable to the housing or housing component by way of the first connecting portion. With other examples the connector, in particular its first connecting portion, may be integrated into the housing or housing component of the injection device. In either way and when connected to the housing or housing component the second connecting portion of the connector may protrude radially outwardly from the housing or housing component.

The radial direction may extend substantially perpendicular to the longitudinal elongation of the housing or housing component of the injection device. With some examples the radial direction may extend obliquely with regards to the longitudinal direction. Since the injection device comprises the connector as described above all features, effects and benefits as described above in connection with the connector equally apply to the injection device; and vice versa.

According to a further aspect the present disclosure relates to a kit comprising a first injection device and comprising a second injection device. The kit further comprises a connector as described above. The connector may be provided as a separate part in addition to the first injection device and the second injection device. With other examples the connector may be integrated into at least one of the first injection device and the second injection device. With further examples there may be provided two connectors, one of which being attached to or integrated into the first injection device and the other one of which being attached to or integrated into the second injection device.

The first injection device comprises a first housing. The first housing, i.e. the first injection device, comprises at least one housing component. The housing component may be implemented as a protective cap detachably connectable to a dispensing end of the injection device. The housing component may comprise a tubular-shaped body, i.e. a proximal housing portion of the injection device. The body may be sized to accommodate the drive mechanism.

With other examples the housing component comprises the cartridge holder, e.g. configured and sized to accommodate the medicament container, which may be implemented as a medicament cartridge. With further examples the housing component comprises a fastening clip, e.g. provided at or on the protective cap of the injection device.

Like the first injection device also the second injection device comprises a second housing comprising at least one housing component. With some examples the first injection device and the second injection device may be somewhat identical. The first injection device and the second injection device may belong to a common type of injection devices.

The kit further comprises a connector as described above. Insofar, all features, effects and benefits as described above in connection with the connector as well as described above in connection with the injection device equally apply to the kit; and vice versa. Moreover, the first connecting portion of the connector is mechanically connectable to the at least one housing component of the first injection device. Alternatively, the first connecting portion of the connector is integrated into the at least one housing component of the first injection device.

The second connecting portion of the connector is mechanically connectable to the at least one housing component of the second injection device or the second connecting portion of the connector is mechanically connectable to one of the first connecting portion and the second connecting portion of another connector attachable to or integrated into the second injection device. Here, the connector of the kit and the another connector attachable to or integrated into the second injection device may be either identically or differently-shaped. With some examples also the another connector may belong or may contribute to the kit comprising the first injection device, the second injection device and the connector.

The kit is not limited to only two injection devices. The number of injection devices contributing to the kit may be as large as 4, 8, 12, 16, 20 or even more. With some examples and when the connector is integrated into a housing component of an injection device the kit may comprise an equal amount of injection devices and connectors. Hence, there may be provided one connector per injection device.

With further examples and when the connector is provided as a separate part in addition to the first injection device and the second injection device the kit may comprise a number of injection devices and a number of connectors, wherein the number of connectors equals the number of injection devices minus one.

With some configurations of a kit, the first injection device may be connected to the second injection device by way of the connector. The second injection device may be connected to a third injection device by way of a second connector and so on. The injection devices of the kit may form or contribute to an array of injection devices, mutually connected by respective connectors. Such an array of injection devices may require less storage space and may provide a well-defined collision-less arrangement and assembly of a rather large number of injection devices.

With other examples the kit comprises first and second housing components of an injection device rather than first and second completely equipped injection devices. In this way, individual and identical housing components, such as a proximal body of a housing or a distally located protective cap of a housing of an injection device can be mutually attached or connected to each other before the injection device is finally assembled.

Hence, identically shaped protective caps, each provided with a connector could be mutually connected by the connector and could be transported to a final assembly stage in an automated assembly line during the process of injection device assembly. In this way, logistics and expenditure for handling individual parts of an injection device, in particular of rather spacious parts of an injection device, namely housing component of an injection device, could be facilitated and simplified.

Insofar, any reference of connection of a connecting portion to an injection device may equally apply to a connection of such a connecting portion to a housing component of such injection devices; and vice versa.

With further examples, the kit also comprises an auxiliary device or add-on device configured to interact with at least one of the first and second injection devices. As described above, at least one of the first and the second injection devices can be replaced by the add-on device or auxiliary device. Here, the connector may provide a mutual mechanical fastening and interconnection of an injection device with an add-on device or auxiliary device.

According to a further aspect the present disclosure relates to a method of interconnecting a first injection device and a second injection device. The method comprises the steps of integrating a connector as described above into a housing or housing component of the first injection device or alternatively, attaching such a connector to a housing or housing portion of the first injection device. In a subsequent method step the second connecting portion of the connector is connected with a housing or housing portion of the second injection device. Upon establishing such a connection the first and the second injection devices are mutually interconnected by way of the connector.

Alternatively, and instead of connecting the second connecting portion with a housing or housing component of the second injection device the second connecting portion of the connector is connected to one of the first connecting portion and the second connecting portion of another connector attached to or integrated into the second injection device. Here, the first connector and the second connector may comprise a male and a female connecting part that are complementary shaped so as to detachably engage.

Typically, the method as described herein is to be implemented by making use of a connector as described above and/or by making use of an injection device as described above. Insofar, all features, effects and benefits as described above in connection with the connector, the injection device and the kit equally apply to the method of interconnecting first and second injection devices; and vice versa.

Of course, the method is not limited to the mutual interconnection of first and second injection devices. There may be interconnected a large number of injection devices, e.g. of up to 4, up to 8, up to 12 or up to 16 or even more injection devices, thereby forming a kit as described above.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N—(ω-carboxyheptadecanoyl)-des (B30) human insulin and B29-N—(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device or injection device may involve a needle-based injection system as described in Table 1 of section 5.2 of ISO 11608-1: 2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically illustrates one example of an injection device,

FIG. 2 schematically illustrates one example of a kit comprising three injection devices and two connectors,

FIG. 3 schematically shows one example of the connector of FIG. 2 in a separate perspective illustration,

FIG. 4 shows the connector according to FIG. 3 from above,

FIG. 5 shows the connector of FIGS. 3 and 4 in a side view,

FIG. 6 shows another example of a connector in perspective illustration,

FIG. 7 shows the connector of FIG. 6 from above,

FIG. 8 shows an example comprising two housing portions and two connectors,

FIG. 9 shows another example of a connector in perspective illustration,

FIG. 10 shows the connector of FIG. 9 from above,

FIG. 11 shows an example of a kit with three housing components mutually interconnected by a number of connectors according to FIGS. 9 and 10,

FIG. 12 schematically illustrates another example of a connector in a first perspective illustration,

FIG. 13 shows the connector of FIG. 12 in another perspective illustration,

FIG. 14 shows first and second housing components interconnected,

FIG. 15 shows the configuration of FIG. 14 as seen from a distal longitudinal end,

FIG. 16 shows another example of a connector in a first perspective illustration,

FIG. 17 shows the connector of FIG. 16 in another perspective illustration,

FIG. 18 shows two housing components mutually interconnected,

FIG. 19 shows the assembly of FIG. 18 as seen from a distal longitudinal end,

FIG. 20 shows a further example of housing components connected by mutually engaging connectors,

FIG. 21 shows a front view of the housing components before mutually engaged, and

FIG. 22 illustrates a flowchart of a method of interconnecting first and second injection devices.

### DETAILED DESCRIPTION

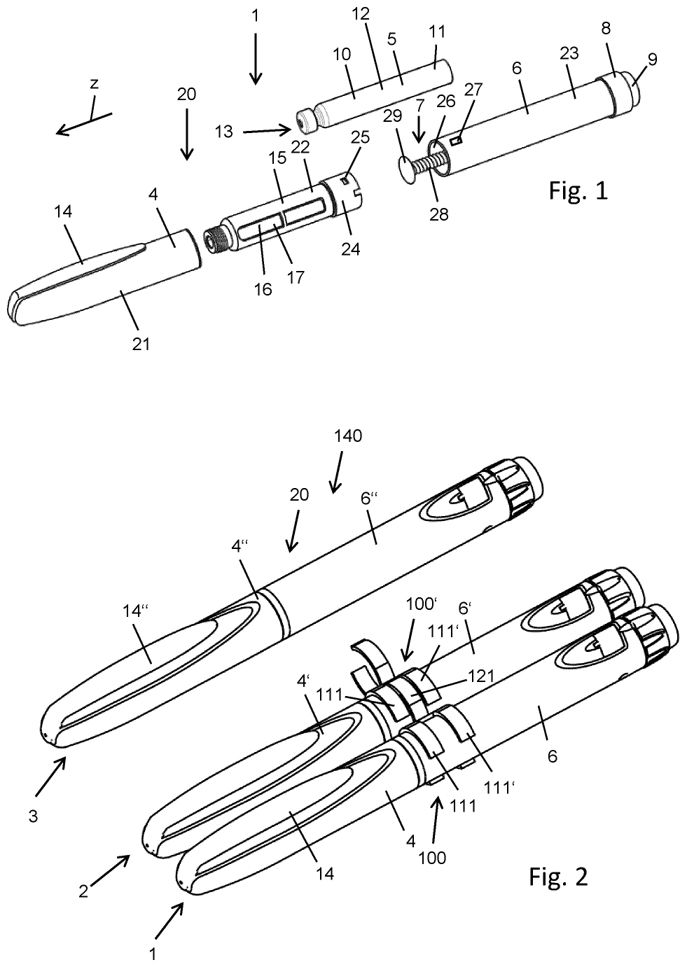

FIG. 1 illustrates one example of a drug delivery device 1, which is implemented as a handheld injection device or as a pen-type injector. Typically, the drug delivery device 1 comprises an elongated shape. It may extend along a longitudinal direction (z). Towards a longitudinal distal direction the drug delivery device 1 comprises an outlet for dispensing a medicament. Towards a proximal end the drug delivery device 1 comprises at least one of a dose member 8 and a trigger 9, by way of which a dose of equal or individual and different size can be set and dispensed, respectively. The drug delivery device 1 comprises a housing 20. The housing 20 as well as the drug delivery device 1 may comprise numerous device components. For instance, the drug delivery device 1 may comprise a protective cap 4 covering a distal housing component configured as a cartridge holder 15.

The housing 20 may further comprise a body 6, e.g. forming a proximal end of the housing 20. The body 6 is configured to house a drive mechanism 7. The body 6 max comprise a tubular shaped sidewall 23. Typically, the drive mechanism 7 comprises a piston rod 28, e.g. provided with a pressure piece 29 located at its distal end. The cartridge holder 15 is sized and configured to accommodate a medicament container 5. In the present example the medicament container 5 comprises a cartridge 10. The cartridge 10 comprises a tubular-shaped barrel 11 containing the liquid medicament 12. Towards a distal end the cartridge 10 comprises a dispensing end 13, which may be closed and covered by a seal. The seal may be pierceable by a double-tipped injection needle. With some examples the injection needle (not illustrated) is connectable to a distal end of the cartridge holder 15. The distal end of the cartridge holder may comprise a threaded socket to engage with a complementary shaped part of the injection needle or needle assembly.

The distal end of the cartridge holder 15 may comprise a through opening to receive a distally pointing portion of the double-tipped injection needle configured to pierce and to penetrate the seal of the cartridge 10. With other examples (not illustrated) the cartridge 10 or medicament container 5 may be readily equipped with an injection needle. Typically, a proximal end of the medicament container 5 is sealed by a movable stopper. The stopper is movable along the longitudinal direction inside the barrel under the action of the advancing piston rod 28 thereby expelling a dose of the medicament 12 from the medicament container 5.

As particularly illustrated in FIG. 11 the cartridge holder 15, hence a sidewall 22 of the cartridge holder 15 comprises a window 16 implemented as an aperture 17 and hence as a through opening extending through the sidewall 22.

A proximal end of the cartridge holder 15 is implemented as a proximal connecting end configured to engage with a distal connecting end 26 of the body 6. This way, the proximally located connecting end 24 of the cartridge holder 15 can be connected to a distally located connecting end 26 of the body 6. The connecting end 24 is provided with a fastening element 25 complementary shaped to a counter fastening element 27 of the body 6. With some examples the proximal connecting end 24 of the cartridge holder 15 comprises a radially stepped down insert configured for longitudinal insertion into a distally located receptacle formed by the sidewall 23 of the body 6.

The presently illustrated example of a drug delivery device 1 is only exemplary for a large variety of injection devices 1 that could be manufactured in a mass manufacturing process and hence in an automated production line. The drug delivery device 1 may comprise a fixed dose injection device, a variable dose injection device, an autoinjector, an all-mechanically implemented pen-type injector, with or without a movable dial extension or an electronically or semi-electronically implemented injection pen.

With some examples a cartridge 10 or medicament container 5 readily inserted in the cartridge holder 15 may form or constitute a first subassembly. The drive mechanism 7 mounted inside the body 6 may form a second subassembly. When implemented as a reusable injection device 1 the cartridge holder 15 is detachably connected to the body 6. When implemented as a disposable device the cartridge holder 15 is non-detachably, hence durably connected to the body 6. The cartridge 10 filled with the liquid medicament 12 is readily assembled inside the housing 20 and may thus require to store the entirety of the injection device 1 in a refrigerated area.

In FIGS. 2-5 one example of a connector 100 is illustrated in connection with three injection devices 1, 2, 3. The injection devices 1, 2, 3 are each implemented as a pen-type injector. The injection devices 1, 2, 3 each comprise a housing 20 of elongated tubular shape. Each housing 20 comprises a protective cap 4 configured to cover the dispensing end of the injection device 1, 2, 3. Each housing 20 further comprises a body 6 forming or constituting a proximal housing component of the injection device 1, 2, 3.

As illustrated, the first injection device 1 comprises a protective cap 4 detachably connected to a cartridge holder 15 and/or to the body 6, e.g. by a clip fastener or screw type connection. The protective cap 4 comprises a sidewall 21 of substantially tubular shape comprising a receptacle open in proximal direction and sized to receive the cartridge holder therein. A clip fastener or a screw-type connector of the protective cap 4 is typically provided at or near a proximal end of an inside section of the sidewall 21.

The second injection device 2 comprises an identically shaped protective cap 4′ and a rather identically shaped body 6′. The third injection device 3 comprises a distally located protective cap 4″ and a proximally located body 6″. The protective cap 4 comprises a fastening clip 14, allowing to detachably fix the protective cap 4 and hence the entire injection device 1 to a piece of garment.

FIG. 2 illustrates one example of a kit 140 comprising a first injection device 1, a second injection device 2, a third injection device 3 and further comprising a first connector 100 and a second connector 100′. By way of the first connector 100 the first injection device 1 is connected to the second injection device 2. By way of the second connector 100′ the second injection device 2 is connectable to the third injection device 3.

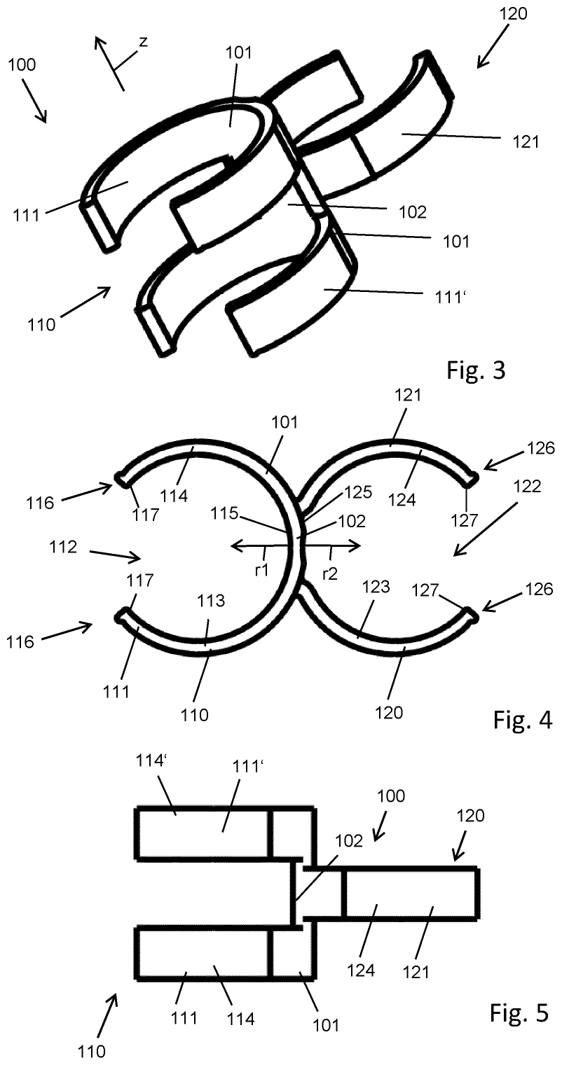

In FIGS. 3-5 the connector 100 is shown in greater detail. The connector 100 comprises a body 101 with a base portion 102. The body 101 and hence the connector 100 comprises a first connecting portion 110 and a second connecting portion 120. Both, the first connecting portion 110 and the second connecting portion 120 comprise a fastening bracket 111, 121. The fastening bracket 111 comprises a receptacle 112 sized and shaped to receive a portion of the body 6. Likewise, the second connecting portion 120 comprises a fastening bracket 121 forming or constituting a further receptacle 122 sized and shaped to receive the body 6′.

The fastening bracket 111 comprises a first arm portion 113 and a second arm portion 114, which are both of arcuate shape. The arm portions 113, 114 are interconnected by a middle section 115 e.g. coinciding with the base portion 102, which is located in the center or in an overlapping section of the first connecting portion 110 and the second connecting portion 120. The arm portions 113, 114 may comprise an arcuate or (semi-) circular structure. They may extend in circumferential direction from the middle section 115.

The arm portions 113, 114 each comprise a free end section 116 facing away the middle section 115. The free end section 116 is oriented radially inwardly with regards to the receptacle 112. Accordingly, the free end section 116 may form or may comprise a flange portion 117 by way of which the body 6 can be fastened in radial direction inside the receptacle 112.

The arm portions 113, 114 provide or form a fastening bracket 111 of annular shape. The fastening bracket 111 is an open ring structure. The aperture provided between the oppositely located free ends 116 of the first and the second arm portions 113, 114 is sized to receive the tubular-shaped sidewall of the body 6 in radial direction. Hence, the circumference of the body 6, i.e. the outside facing portion of the sidewall of the body 6 can be inserted through the through opening between the free ends 116 in radial direction, thereby pivoting or elastically deforming the arm sections 113, 114 radially outwardly so as to at least temporally enlarge the free space between the free end sections 116 until the body 6 reaches the final assembly configuration as illustrated in FIG. 2.

The fastening bracket 121 of the second connecting portion 120 is quite similarly shaped compared to the fastening bracket 111 of the first connecting portion 110. The fastening bracket 121 is also of annular shape. It forms or constitutes a semicircular structure. It comprises a first arm portion 123 and a second arm portion 124. The arm portions 124, 123 are mutually connected by a middle section 125. The middle section 125 may longitudinally overlap with the middle section 115. The middle sections 115, 125 belong or may constitute the base portion 102 located radially between the first connecting portion 110 and the second connecting portion 120. The free end sections 126 of the first and second arm portions 123, 124 facing away the middle section 125 each comprise or form an inwardly directed flange portion 127 so as to at least partially enclose or clasp around the tubular shaped body 6′.

As it is further apparent from the illustrations of FIGS. 3 and 5 the first connecting portion 110 comprises a first fastening bracket 111 and a second fastening bracket 111′. The first and the second fastening brackets 111, 111′ are separated by a longitudinal distance. The longitudinal distance may be defined by the longitudinal extent of the fastening bracket 121 and/or by the longitudinal extent of the second connecting portion 120. The second fastening portion 120 is arranged longitudinally between the first and second fastening brackets 111, 111′ of the first connecting portion 110.

The fastening bracket 111 and the fastening bracket 111′ are somewhat identical in shape. They may overlap as seen in a longitudinal projection as shown in FIG. 4. By way of two fastening brackets 111, 111′ of the first connecting portion 110 there can be provided a rather stable and robust connection of the connector 100 to the first injection device 1. A break loose force for detaching the first injection device 1 from the connecting portion 110 may be somewhat larger than a break loose force required for detaching the second injection device 2 from the second fastening or connecting portion 120.

As further illustrated in FIG. 4 the connecting portion 110 and hence the first fastening bracket 111 extends or protrudes radially from the base portion 102 by way of which the first and the second connecting portions 110, 120 are interconnected. The second fastening portion 120 and hence the second fastening bracket 121 extends along a second radial direction r2 and hence opposite to a first radial direction r1, into which the first connecting portion 110 extends. Insofar, the first and second connecting portions 110, 120 extend or protrude in opposite radial directions r1, r2 as seen from the base portion 102.

The fastening brackets 111, 111′ of the first connecting portion 110 are separated in longitudinal direction by a distance that is governed by the respective longitudinal extent of the second connecting portion 120. In this way, and as illustrated in the mutual assembly configuration of the kit 140 of FIG. 2, there can be provided a somewhat nested or interleaved engagement configuration when numerous connectors 100, 100′ are used to mutually interconnect a number of injection devices 1, 2, 3.

As illustrated, the second connecting portion 120 and hence the second fastening bracket 121 of the first connector 100 can be arranged longitudinally between the fastening brackets 111, 111′ of the further connector 100′. Likewise, the longitudinal separation of the fastening brackets 111, 111′ clasping around the body 6 of the first injection device 1 provide assembly space therebetween for receiving a fastening bracket 121 of a connecting portion 120 of a further connector 100, e.g. connected with its first connecting portion 110 to another injection device (not illustrated).

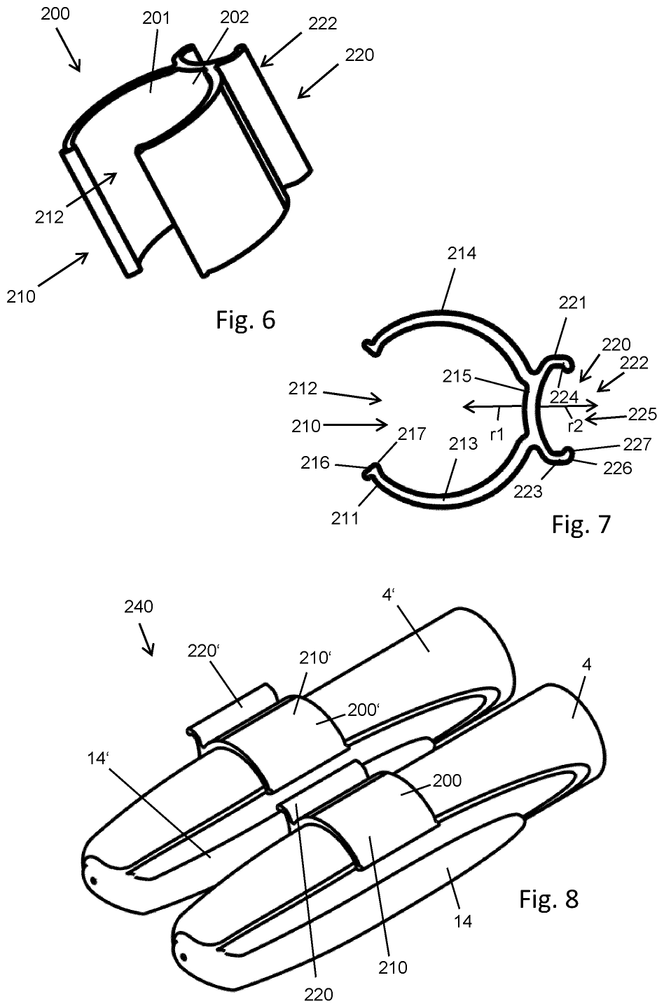

In FIGS. 6-8 there is shown another example of a connector 200. This connector 200 also comprises a first connecting portion 210 and a second connecting portion 220. First and second connecting portions 210, 220 are integrally formed and belong to a common body 201. The first connecting portion 210 extends along a first radial direction r1 from a base portion 202. The second connecting portion 220 extends along a second radial direction r2 from the base portion 202. Here and in contrast to the example of FIGS. 2-5 the geometry and shape of the first connecting portion 210 distinguishes from the geometry and shape of the second connecting portion 220.

The connecting portions 210, 220 each comprise a fastening bracket 211, 221. Each fastening bracket 211, 221 comprises or forms a receptacle 212, 222. The receptacle 212 of the first connecting portion 210 is configured to receive a sidewall of a protective cap 4. The receptacle 222 of the second connecting portion 220 is sized to receive another housing component of the injection device 1. In particular, the receptacle 222 of the second connecting portion 220 is sized and shaped to receive a fastening clip 14 of a protective cap 4 of the injection device 1.

The receptacle 222 also comprises a first arm portion 223 and a second arm portion 224. The first and the second arm portions 223, 224 comprise an inwardly extending flange portion 227 at their free end sections 226.

Here, the first connecting portion 210 is sized and configured to attach the connector 200 to a housing component, e.g. to a protective cap 4 of the injection device 1. The second connecting portion 220 provides a receptacle 222 forming or constituting a female connecting part 225, which is configured to engage with a male connecting part, e.g. formed by the fastening clip 14 of the housing component, e.g. of the protective cap 4.

In this way and with the kit 240 of FIG. 8 the connector 200 provides a mutual connection of housing component 4, 4′ of injection devices 1, 2. Here, the kit 240 is only illustrated partially. Of course, since the protective cap 4 of a first injection device 1 is connected to a protective cap 4′ of a second injection device 2 the connector 200 may not only provide a mutual connection between individual housing components 4, 4′ but may also provide a durable and stable mutual fastening and interconnection between the drug delivery devices 1, 2 equipped with such housing components 4, 4′.

The fastening bracket 211 also comprises first and second arm portions 213, 214. The arm portions 213, 214 each comprise a free end section 216 equipped or provided with an inwardly extending flange portion 217 or forming such an inwardly extending flange portion 217 so as to provide a positive fastening to the housing component 4.

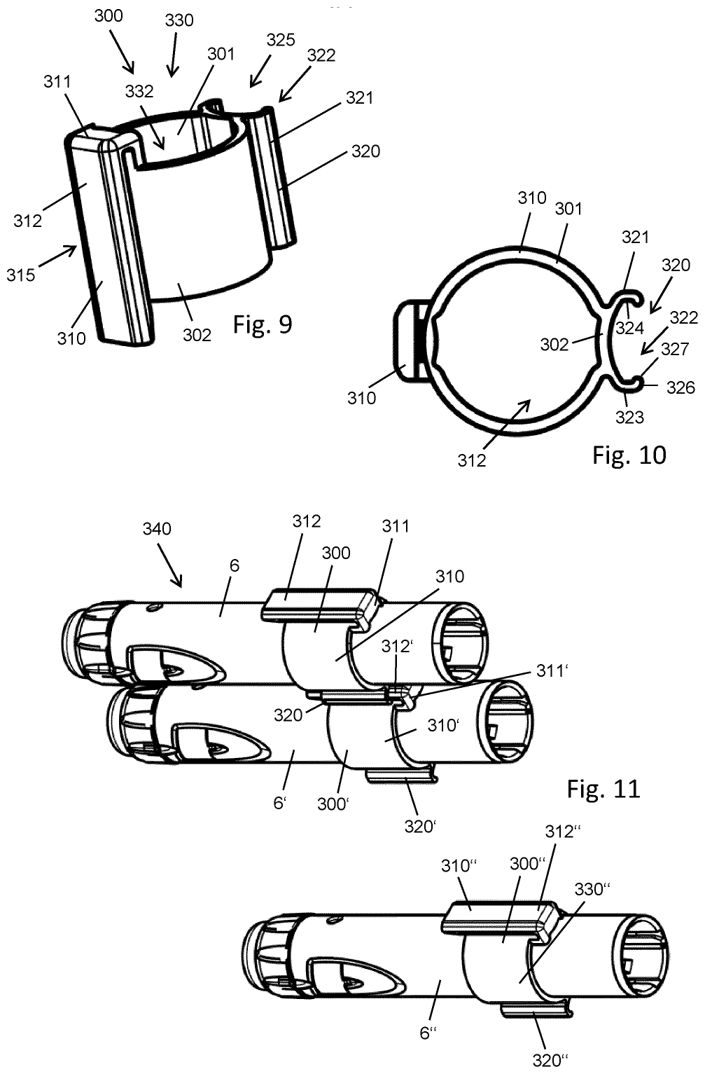

In the further example of a connector 300 as illustrated in FIGS. 9-11 the connector 300 also comprises a somewhat tubular shaped body 301 with a tubular and/or annular shaped base portion 302. Here, the base portion 302 comprises or forms a third connecting portion 330 configured to detachably connect to a housing 20 or housing portion 4, 6, 15 of the injection device 1. The connector 300 and the injection devices 1,2 form another kit 340.

The base portion 302 may comprise a tubular or cylindrically-shaped receptacle 332 to clasp around the body 6 of the injection device 1. The interior of the receptacle 332, hence the interior of the sidewall forming the receptacle 332 is sized to engage with an outside surface of the sidewall of the body 6 as illustrated in FIG. 11. With the connector 300 the first connecting portion 310 protrudes radially outwardly from an outside of the base portion 302. The second connecting portion 320 protrudes radially outwardly from another portion of the base portion 302. Typically, the first connecting portion 310 and the second connecting portion 320 are located at diametrically opposite sides on the outside surface of the base portion 302.

The second connecting portion 320 comprises a fastening bracket 321 forming a receptacle 322 to receive and insert portion 312 of the first connecting portion 310 of another connector 300′. The insert portion 312 protrudes radially outwardly from an outside surface of the cylindrically shaped base portion 302. It comprises an elongated profile extending parallel to the longitudinal direction (z). It is rigidly connected to the base portion 302 by a radially outwardly extending bridging portion 311. The insert portion 312 forms a male connecting part 315 complementary shaped to the inside geometry or inside structure of the receptacle 322 of the fastening bracket 321. Here, the receptacle 322 forms or constitutes a female connecting part 325 complementary shaped to the male connecting part 315.

The insert portion 312 is of substantially oblong shape. It extends in longitudinal direction and comprises a constant cross-section or profile as seen in longitudinal direction. It is complementary shaped to the inside facing structure of the receptacle 322. The receptacle 322 comprises first and second oppositely located arm portions 323, 324. The arm portions 323, 324 comprise an inwardly extending flange portion 327 at their free ends 326 thereby forming a c-shaped arm portion by way of which the oppositely located circumferential edges of the insert portion 312 can be enclosed and hence fixed with regard to the radial and circumferential direction when the insert portion 312 has been inserted in longitudinal direction into the receptacle 322.

By way of the connector 300 a housing portion, e.g. a tubular housing portion 6 of the housing 20, which is void of any protrusions or recesses can be retrofitted with a connecting structure by way of which another housing component, e.g. another body 6′ equally equipped with such a connector 300 can be connected to a connector 300′ as illustrated in FIG. 11. The inside surface of the sidewall of the base portion 302 and hence the sidewall of the receptacle 332 may frictionally engage with the outside surface of the tubular shaped body 6. Another connector 300′ may accordingly engage with another body 6′. Now and for the bodies 6, 6′, each of which being equipped with a suitable connector 300, 300′, there can be provided a mutual interconnection between the connectors 300, 300′, and hence between the bodies 6, 6′, namely by engaging the first connecting portion 310′ of the connector 300′ with the second connecting portion 320 of the connector 300.

Likewise, a first connecting portion 310″ of another connector 300″ connected with a further body 6″ can be connected to the connecting portion 320′ of the connector 300′, which is connected to the body 6′.

The connector 300 provides retrofitting of arbitrary housing components 6 of a housing 20 of an injection device 1. Housing components 6, 6′, 6″ each of which being equipped with a connector 300, 300′, 300″ can be mutually and detachably interconnected.

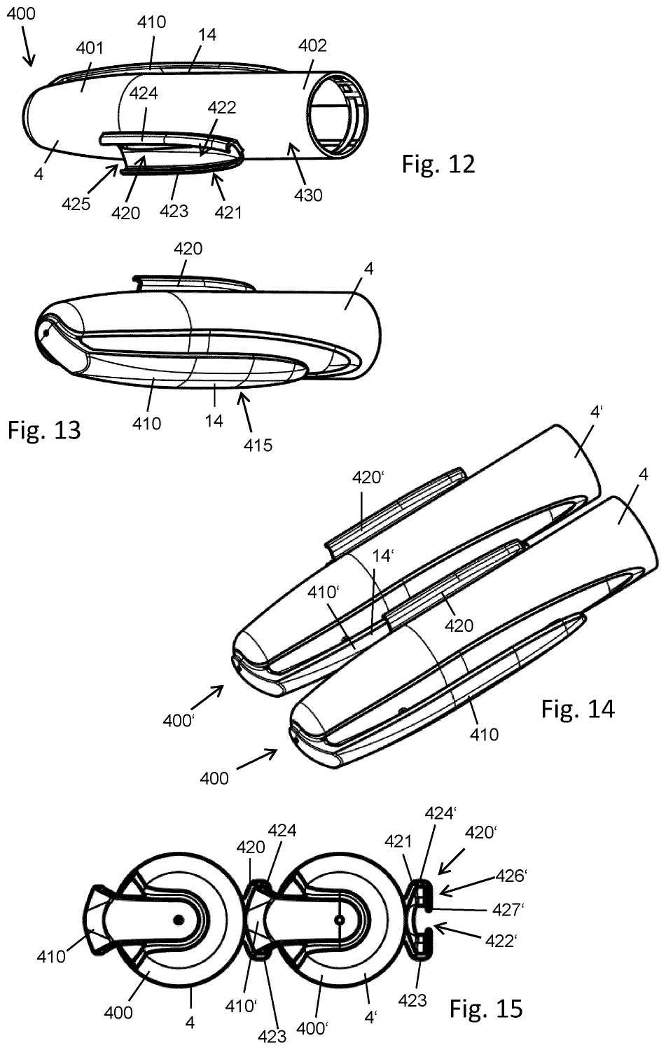

With the further example as illustrated in FIG. 12-15 a connector 400 is integrally formed with a housing portion or housing component, namely with a protective cap 4 of the injection device 1. Here, the tubular shaped protective cap 4 forms or constitutes the body 401 and the base portion 402 of the connector 400. A first connecting portion 410 is implemented or coincides with the fastening clip 14. The first connecting portion 410 protrudes radially outwardly from the tubular shaped body 401 or base portion 402. With some examples, the base portion 402 comprises or forms a third connecting portion 430 configured to detachably connect to a housing 20 or housing portion 4, 6, 15 of the injection device 1.

The second connecting portion 420 is located at a diametrically opposite side of the connector 400. The second connecting portion 420 comprises a fastening bracket 421 provided with a receptacle 422. Also here, the receptacle 422 is provided with a first arm portion 423 or sidewall portion and a second arm portion 424 forming or constituting a second sidewall protruding radially from the outside surface of the tubular sidewall of the base portion 402.

As further illustrated in FIGS. 12 and 15 the free end sections 426 of the arm portion 423, 424 comprise an inwardly directed flange portion 427 to grip under the oblong shaped first connecting portion 410. Here, the second connecting portion 420 comprises a female connecting part 425 providing a recessed structure in form of a receptacle 422, which is open towards the distal end of the protective cap 4. Insofar, the protective cap 4′ provided with the fastening clip 14′ forming the first connecting portion 410′ in form of a male connecting portion 415 can be inserted in proximal longitudinal direction into the receptacle 424 provided by the fastening bracket 421. In this way, the protective caps 4, 4′ as illustrated in FIGS. 14 and 15 can be mutually connected and attached to each other.

As further illustrated in FIG. 12, the receptacle 422 delimited or confined in circumferential direction by the arm portions 423, 424 tapers towards the proximal direction so as to provide a longitudinal end stop for the first connecting portion 410 when longitudinally inserted into the receptacle 422 in proximal direction. Here, a correspondingly tapered profile of the first connecting portion 410, i.e. of the fastening clip 14 may provide a clamping effect or may frictionally engage with the second connecting portion 420 when reaching a final assembly configuration as illustrated in FIGS. 14 and 15.

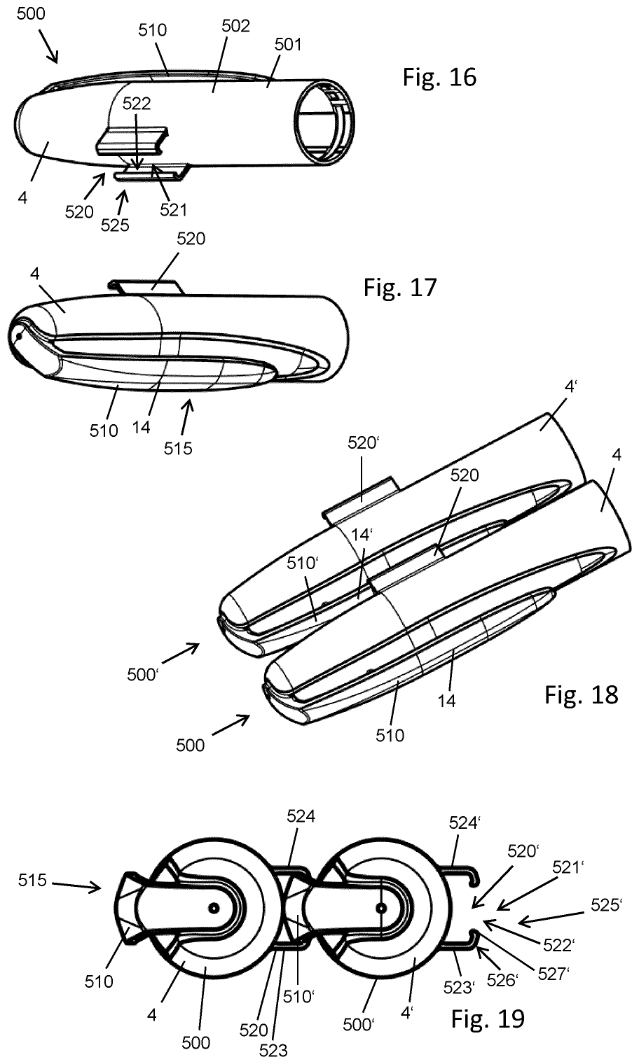

The further example as illustrated in FIGS. 16-19 is functionally similar to the example as illustrated in FIGS. 12-15. Here, the connector 500 also comprises a body 501 coinciding with the protective cap 4 of the injection device 1. The body 501 comprises a base portion 502 featuring a somewhat tubular-shaped sidewall to receive the distally located dispensing end of the injection device 1. The base portion 502 forms or constitutes a receptacle sized to receive the cartridge holder 15 of the injection device 1.

Also here, the connector 500 comprises a first connecting portion 510 implemented as a male connecting part 515 protruding radially outwardly from the body 501. the first connecting portion 510 is provided and formed by the fastening clip 14 of the protective cap 4. Diametrically opposite on the outside surface of the base portion 502 or body 501 there is provided the second connecting portion 520 comprising a fastening bracket 521 defining a receptacle 522 complementary shaped to the cross-section and shape of the first connecting portion 510. Here, the receptacle 522 forms a female connecting part 525. Also here, the receptacle 522 is confined in circumferential direction by a first arm portion 523 and a second arm portion 524. The arm portions 523, 524 are provided with an inwardly extending flange portion 527 at their free end section 526. In this way and when the first connecting portion 510′ of a second connector 500′ is inserted into the second connecting portion 520 of a first connector 500 there can be provided a rather rigid and robust mutual connection between the connectors 500, 500′ and hence between the respective housing components, e.g. in form of the protective caps 4, 4′.

The arm portions 523, 524 of the second connecting portion 520 comprise a L-shaped profile as seen in the transverse direction. The flange portions 527 of the arm portions 523, 524 face towards each other. They are of oblong shape and comprises a somewhat constant profile as seen in longitudinal direction.

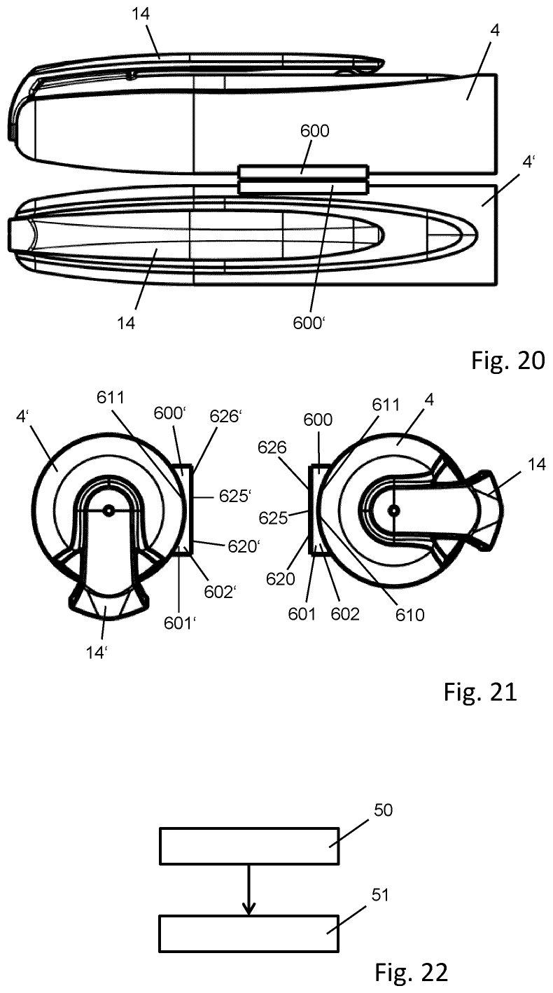

With the further example of FIGS. 20 and 21 the connector 600 also comprises a connector body 601 and a base portion 602. Here, the connecting portion 610 is provided on one side of the base portion 602 and the second connecting portion 620 is provided on an opposite side of the base portion 602. The first connecting portion 610 is provided with a connecting surface 611 configured to make contact with an outside surface of the housing component 4. There is provided another connector 600′ equally or identically shaped to the connector 600. The further connector 600′ is attached to another housing component 4′. The further connector 600′ comprises a body 601′ with a base portion 602′. Also here, the first connecting portion 610′ and the second connecting portion 620′ are located on opposite sides of the base portion 602′.

The connecting portions 610, 610′ may be provided with a connecting surface 611, 611′. The connecting surfaces 611, 611 may comprise an adhesive or an adhesive layer, by way of which the connectors 600, 600′ can be detachably or durably, i.e. non-detachably, fixed to an outside facing portion of the housing component 4, 4′, which in the presently illustrated example is implemented as a protective cap 4, 4′.

The second connecting portion 620, 620′ comprises a connecting surface 625, 625′. The connecting surface 625 of the first connector 600 is shaped and/or configured to detachably connect or to detachably fix to the counterpart connecting surface 625′ of the further connector 600′ connected to the second housing component 4′. With some examples the connecting surfaces 625, 625′ may comprise mutually corresponding hook and loop fasteners that allow a detachable and hence releasable mutual interconnection between the connecting surfaces 625, 625′.

In a further example the body 601 and/or the base portion 602 of the connector 600 comprise a pliable material, e.g. an adhesive tape being provided with a hook and loop fastener on an outside surface so as to provide a mutual interconnection between first and second connectors 600, 600′ when attached to respective housing components 4, 4′ of injection devices 1, 2.

With some examples, the body 601 may comprise an arcuate shape connecting surface 611 at or on the first connecting portion 610. The second connecting portion 620 may comprise a rather flat and planar-shaped connecting surface 625. In this way, the connecting surface 611 of the connecting portion 610 is particularly configured to attach to a tubular shaped outside surface of a sidewall of a housing portion 4, 6. The rather flat and planar-shaped connecting surface 625 of the second connecting portion 620 may provide a comparatively large contact surface to make contact with a respective counter contact surface 625′ of another connector 600 connected or connectable to another housing portion 4′, 6′ of a further injection device 2.

The flowchart of FIG. 22 is illustrative of the method of mutually interconnecting the first and the second injection devices 1, 2. In a first step 50 a connector 100 as described above is integrated into a housing or housing component 4, 6 of the first injection device 1, or alternatively, the connector 100 is attached to a housing or housing portion 4, 6 of the first injection device 1, e.g. to a protective cap 4.

In a subsequent method step 51 the second connecting portion 120 of the connector 100 is connected with a housing or housing portion 4′ of the second injection device 2. Upon establishing such a connection the first and the second injection devices 1, 2 become mutually interconnected by way of the connector 100.

Alternatively, and instead of connecting the second connecting portion 120 with a housing or housing component of the second injection device 2 the method step 51 may be executed by connecting the second connecting portion 320, 420, 520 of the connector 300, 400, 500 to a first connecting portion 310, 410, 510 of another connector 300′, 400′, 500′ attached to or integrated into the second injection device 2. Here, the first connector 300, 400, 500 and the second connector 300′, 400′, 500′ may comprise a male and a female connecting part that are complementary shaped so as to detachably engage.

### REFERENCE NUMBERS

1 injection device
2 injection device
3 injection device
4 protective cap
5 medicament container
6 body
7 drive mechanism
8 dose member
9 trigger
10 cartridge
11 barrel
12 medicament
13 dispensing end
14 fastening clip
15 cartridge holder
16 recess
17 aperture
20 housing
21 sidewall
22 sidewall
23 sidewall
24 connecting end
25 fastening element
26 connecting end
27 fastening element
28 piston rod
29 pressure piece
100 connector
101 body
102 base portion
110 connecting portion
111 fastening bracket
112 receptacle
113 arm portion
114 arm portion
115 middle section
116 free end section
117 flange portion
120 connecting portion
121 fastening bracket
122 receptacle
123 arm portion
124 arm portion
125 middle section
126 free end section
127 flange portion
140 kit
200 connector
201 body
202 base portion
210 connecting portion
211 fastening bracket
212 receptacle
213 arm portion
214 arm portion
215 middle section
216 free end section
217 flange portion
220 connecting portion
221 fastening bracket
222 receptacle
223 arm portion
224 arm portion
225 female connecting part
226 free end section
227 flange portion
240 kit
300 connector
301 body
302 base portion
310 connecting portion
311 bridging portion
312 insert portion
315 male connecting part
320 connecting portion
321 fastening bracket
322 receptacle
323 arm portion
324 arm portion
325 female connecting part
326 free end section
327 flange portion
330 connecting portion
332 receptacle
340 kit
400 connector
401 body
402 base portion
410 connecting portion
415 male connecting part
420 connecting portion
421 fastening bracket
422 receptacle
423 arm portion
424 arm portion
425 female connecting part
426 free end section
427 flange portion
430 connecting portion
500 connector
501 body
502 base portion
510 connecting portion
515 male connecting part
520 connecting portion
521 fastening bracket
522 receptacle
523 arm portion
524 arm portion
525 female connecting part
526 free end section
527 flange portion
600 connector
601 body
602 base portion
610 connecting portion
611 connecting surface
620 connecting portion
625 connecting surface
626 hook-and-loop fastener

1. A connector (100) for establishing a mechanical connection between a first injection device (1) and a second injection device (11), wherein the first injection device (1) and the second injection device (11) comprises a housing (20) of elongated shape extending in a longitudinal direction (z), the connector (100) comprising:
a body (101; 201; 301; 401; 501; 601),
a first connecting portion (110; 210; 310; 410; 510; 610) connectable to the first injection device (1) or integrated into the first injection device (1),
a second connecting portion (120; 220; 320; 420; 520; 620) detachably connectable to the second injection device (2).

2. The connector (100) according to claim 1, wherein the first connecting portion (110; 210; 310; 410; 510; 610) and the second connecting portion (120; 220; 320; 420; 520; 620) are rigidly fastened to one another.

3. The connector (100) according to any one of the preceding claims, wherein the body (101; 201; 301; 401; 501) is a unitary body and wherein the first connecting portion (110; 210; 310; 410; 510) and the second connecting portion (120; 220; 320; 420; 520) are integrally formed with the body (101; 201; 301; 401; 501).

4. The connector (100) according to any one of the preceding claims, wherein at least one of the first connecting portion (110; 210; 310; 410; 510) and the second connecting portion (120; 220; 320; 420; 520) comprises a fastening bracket (111, 121; 211; 221; 321; 421; 521) configured to mechanically engage with a housing (20) or housing portion (4, 6, 14) of at least one of the first injection device (1) and the second injection device (2).

5. The connector (100) according to claim 4, wherein the fastening bracket (111, 121; 211; 311; 421; 521) is of circular, semicircular or concave shape.

6. The connector (100) according to any one of the preceding claims, further comprising a base portion (102; 202; 302; 402; 502; 602) rigidly fastened to the first connecting portion (110; 210; 310; 410; 510; 610) and to the second connecting portion (120; 220; 320; 420; 520; 620), wherein the first connecting portion (110) extends in a first radial direction (r1) from the base portion (102; 202; 302; 402; 502; 602) with regards to the longitudinal direction (z) and wherein the second connecting portion (120; 220; 320; 420; 520; 620) extends or protrudes in a second radial direction (r2) from the base portion (102).

7. The connector (100; 200; 300; 400; 500; 600) according to claim 6, wherein the base portion (102; 202; 302; 402; 502; 602) is located between the first connecting portion (110; 210; 310; 410; 510; 610) and the second connecting portion (120; 220; 320; 420; 520; 620) and wherein at least one of the first connecting portion (110; 210; 310; 410; 510; 610) and the second connecting portion (120; 220; 320; 420; 520; 620) extends or protrudes radially outwardly from the base portion (102; 202; 302; 402; 502; 606).

8. The connector (300; 400; 500; 600) according to any one of the preceding claims, wherein one of the first connecting portion (310; 410; 510; 610) and the second connecting portion (320; 420; 520; 620) comprises a male connecting part (315; 415; 515; 615) configured to detachably engage with a complementary shaped female connecting part (325; 425; 525; 625) of another one of the first connecting portion (310; 410; 510; 610) and the second connecting portion (320; 420; 520; 620) of another connector (300′; 400′; 500′; 600′).

9. The connector (300; 400; 500) according to any one of the preceding claims 6-8, wherein the base portion (302; 402; 502) comprises a third connecting portion (330; 430; 530) configured to detachably connect to a housing (20) or housing portion (4, 6, 15) of an injection device (1) and wherein the first connecting portion (310; 410; 510) is configured to engage with the second connecting portion (320; 420; 520) of another connector (300′; 400′; 500′).

10. The connector (300; 400; 500) according to any one of the preceding claims, wherein the body (401; 501) is integrated into a housing (20) or housing portion (4, 6) of the injection device (1).

11. The connector (300; 400; 500) according to any one of the preceding claims, wherein the body (401; 501) is integrated into a protective cap (4) of an injection device (1) and wherein the first connection portion (410; 510) is integrated into a fastening clip (14) of the protective cap (4).

12. The connector (600) according to any one of the preceding claims, wherein the second connecting portion (620) comprises a connecting surface (625) complementary to a counter connecting surface (625′) of a second connecting portion (620′) of another connector (600) and detachably connectable to the counter connecting surface (625′).

13. An injection device (1) configured for setting and dispensing of a dose of a medicament, the injection device (1) comprising:
a housing (20) configured to receive a medicament container (15) containing the medicament and comprising at least one housing component (4, 6, 14),
a drive mechanism (7) operably engageable with the medicament container (15) for expelling or withdrawing a dose of the medicament from the medicament container (15),
a connector (100; 200; 300; 400; 500; 600) according to any one of the preceding claims connectable to one of the housing (20) or to a housing component (4, 6, 14) or integrated into the housing (20) or housing component (4, 6, 14).

14. A kit (140; 240; 340) comprising a first injection device (1), a second injection device (2) and a connector (100; 200; 300; 400; 500; 600) according to any one of the preceding claims 1-12, wherein
the first injection device (1) comprises a first housing (20) comprising at least one housing component (4, 6, 14),
the second injection device (2) comprises a second housing (20′) comprising at least one housing component (4′, 6′, 14′), and
wherein the first connecting portion (110; 210; 310; 410; 510; 610) of the connector (100; 200; 300; 400; 500; 600) is mechanically connectable to the at least one housing component (4, 6, 14) of the first injection device (1) or is integrated into the at least one housing component (4, 6, 14) of the first injection device (1) and
wherein the second connecting portion (120; 220; 320; 420; 520; 620) of the connector (100; 200; 300; 400; 500; 600)
is mechanically connectable to the at least one housing component (4′; 6′; 20′) of the second injection device (2), or
is mechanically connectable to one of the first connecting portion (310′; 410′; 510′) and the second connecting portion (620′) of another connector (300′; 400′; 500′; 600′) attachable to or integrated into the second injection device (2).

15. A method of interconnecting a first injection device (1) and a second injection device (2), the method comprising the steps of:
integrating a connector (100; 200; 300; 400; 500; 600) according to any one of the preceding claims 1-12 into a housing (20) or housing portion (4, 6, 14) of the first injection device (1) or attaching a connector (100; 200; 300; 400; 500; 600) according to any one of the preceding claims 1-12 to a housing (20) or housing portion (4, 6, 14) of the first injection device (1),
connecting the second connecting portion (120; 220; 320; 420; 520; 620) of the connector (100; 200; 300; 400; 500; 600) with a housing (20′) or housing component (4′; 6′; 14′) of the second injection device (2), or
connecting the second connecting portion (120; 220; 320; 420; 520; 620) to one of the first connecting portion (310′; 410′; 510′) and the second connecting portion (620′) of another connector (300′; 400′; 500′; 600′) attached to or integrated into the second injection device (2).
